(19) United States
(12) Patent Application Publication
(10) Pub. No.: US 2010/0234182 A1
(43) Pub. Date: Sep. 16, 2010

(54) NEUROLOGICAL DEVICE

(75) Inventors: Henry B. Hoffman, Charlotte, NC (US); Christopher L. Klett, Davidson, NC (US); Ian D. Kovacevich, Charlotte, NC (US)

Correspondence Address: Nelson Mullins Riley & Scarborough LLP;IP Department, 100 North Tryon Street, 42nd Floor, Charlotte, NC, 28202-4000, US

(73) Assignee: SAEBO, INC., Charlotte, NC (US)

(21) Appl. No.: 12/688,208

(22) Filed: Jan. 15, 2010

Related U.S. Application Data
(60) Provisional application No. 61/144,952, filed on Jan. 15, 2009.

(51) Int. Cl. A63B 21/00; A61F 5/00

(52) U.S. Cl. 482/8; 602/22

(57) ABSTRACT

A neurological device having a forearm support releasably attached to a user's arm, at least one finger sleeve adapted to be releasably attached to at least one finger, at least one tensor strut having a first end releasably coupled to the at least one finger sleeve and an opposite second end coupled to the forearm support, at least one sensor coupled to at least one of the at least one finger sleeve and the at least tensor strut and configured to detect finger movement and generate electrical signals that are indicative of the movement, and a data device coupled to the sensor and configured to receive the electrical signals and calculate at least one of a range of motion of the at least one finger, a speed of movement of the at least one finger, number of repetitions between flexion and extension of the at least one finger and a pressure exerted by the at least one finger during flexion from the electrical signals. The calculated data is used to remotely track user compliance and rehabilitation compliance by a healthcare provider.

### CLAIM OF PRIORITY

The present application claims priority to U.S. Provisional Patent Application Ser. No. 61/144,952, filed Jan. 15, 2009, entitled Neurological Device, the entire disclosure of which is incorporated by reference herein.

### FIELD OF THE INVENTION

The present invention relates to the field of neurological rehabilitation device constructions in general, and more particularly to an electronic enabled neurological rehabilitation device for collecting data and interacting with a computer program.

### BACKGROUND OF THE INVENTION

A dynamic wrist-hand-finger orthosis or splint is generally used for the positioning of an impaired, injured, or disabled wrist, hand, and fingers. Splints come in a variety of designs: static, static progressive, and dynamic that can be low profile or high profile. Most prior art splints are neurological in nature that either holds the hand in a static functional position, or uses a slight dynamic force to position the fingers. None of the known prior art is neurological based and is designed to allow the user to exercise the impaired upper extremity including the wrist, hand, and fingers.

Many people suffering a neurological injury from stroke, cerebral palsy, brain injury, etc., have upper extremity impairments. Many have some shoulder and elbow movements, but are unable to extend their wrist or fingers to grasp an object. This is usually due to hypertonicity, a condition where the flexor or extensor muscles in the upper extremities are spastic and resist positioning. Dynamic splints can be used to offer slight resistance to hold joints in certain positions. An effective dynamic splint designed to be used for hypertonicity must offer enough force to balance the effects of the increased muscle tone. Also current dynamic splints use a variety of finger cuffs to support the digits. These cuffs are not practical when working on a digit affected by hypertonicity, as they move proximal upon closing the fingers, and then have to be repositioned after opening the fingers manually.

Another problem with prior art neurological rehabilitation devices is waning progress tracking since patients often do not or cannot record home progress due to their illness or lack of interest. Moreover, the lack of interest also leads to lapses in compliance and in-home exercise using the device.

Thus, there is a continuing need for a dynamic splint that will address these prior art deficiencies, and provide the user with an improved way to exercise an impaired upper extremity including the wrist, hand and fingers while tracking both compliance and progress with home therapy.

### SUMMARY OF THE INVENTION

The present invention recognizes and addresses disadvantages of prior art constructions and methods, and it is an object of the present invention to provide an improved wheel slip monitoring system. This and other objects may be achieved by a neurological device comprising a forearm support that is configured to be releasably attached to a user's arm, at least one finger sleeve adapted to be releasably attached to at least one finger, at least one tensor strut having a first end releasably coupled to the at least one finger sleeve and an opposite second end coupled to the forearm support, at least one sensor coupled to at least one of the at least one finger sleeve and the at least tensor strut, the at least one sensor configured to detect finger movement and generate electrical signals that are indicative of the movement, and a data device coupled to the sensor. The data device is configured to receive the electrical signals, calculate at least one of a range of motion of the at least one finger, a speed of movement of the at least one finger, number of repetitions between flexion and extension of the at least one finger and a pressure exerted by the at least one finger during flexion, and store the at least one of a range of motion of the at least one finger, a speed of movement of the at least one finger, number of repetitions between flexion and extension of the at least one finger and a pressure exerted by the at least one finger during flexion in memory. The stored data is used to track user compliance and rehabilitation compliance by a healthcare provider.

In yet another embodiment, a plurality of finger sleeves are adapted to be releasably attached to a respective finger of the user, a thumb sleeve is adapted to be releasably attached to the thumb of a user, a plurality of tension struts are each releasably coupled to a respective one of the plurality of finger sleeves and the thumb sleeve and a plurality of sensors are operatively coupled to at least one of the plurality of finger sleeves and the thumb sleeve and the plurality of tensor struts. Each of the plurality of sensors is operatively coupled to the data device.

In still other embodiments, the plurality of finger sleeves is integrally formed with one another to form a partial glove. In other embodiments, the at least one sensor is wirelessly connected to the data device. In yet other embodiments, the at least one sensor is wired to the data device.

In other embodiments, the data device further comprises at least one of a USB port, an SD card slot and an antenna. In yet other embodiments, the finger sleeve is formed from a plurality of segments, and at least one torsion spring coupled to adjacent segments.

In other embodiments, a plurality of couplers releasably attach the at least one tension strut with the at least one finger sleeve. In still other embodiments, the tension strut is formed from one of a carbon fiber rod, a fiber reinforced polymer, a hydraulic piston and an elastomer band.

In still other embodiments, the data device is operatively coupled to a computing device through a data receiver so that the neurological device is used as an input device to the computing device for making data entries and responding to queries. In these embodiments, the computing device may be running a virtual reality program that allows the user to interact with the program by making finger and hand movements with the neurological device.

In yet other embodiments, the tensor strut second end is coupled to the forearm support by a fastener. In these embodiments, the fastener is one of an adjustable buckle, a set of snaps, buttons, zipper and hooks and loops.

In other embodiments, the finger sleeve is configured to extend from a tip of the finger to a point intermediate the finger tip and a distal interphalangeal joint. In these embodiments, a plurality of tension strut slides, positioned intermediate the tension strut first and second ends intermediate the finger sleeve and the forearm support, releasably attached to the user's finger.

In still other embodiments, the apparatus further comprises an air pneumatic connector having a pneumatic port, the tension strut comprising an air passage that is in fluid communication with the finger sleeve and the pneumatic port, wherein the pneumatic port is configured to receive compressed air.

In another embodiments, a hand support section is intermediate the forearm support section and the tensor strut second end. In this embodiment, the hand support section is movable with respect to the forearm support section over a range of angles.

In accordance with a method of collecting rehabilitation compliance and progress data, the method comprises the steps of providing a neurological device having a forearm support that is configured to be releasably attached to a user's arm, at least one finger sleeve adapted to be releasably attached to at least one finger, at least one tensor strut having a first end releasably coupled to the at least one finger sleeve and an opposite second end coupled to the forearm support, at least one sensor coupled to at least one of the at least one finger sleeve and the at least one tensor strut, the at least one sensor configured to detect finger movement and generate electrical signals that are indicative of the movement, and a data device coupled to the sensor. The method further comprises the steps of receiving the electrical signals, calculating at least one of a range of motion of the at least one finger, a speed of movement of the at least one finger, number of repetitions between flexion and extension of the at least one finger, a pressure exerted by the at least one finger during flexion and date and time, storing the at least one of a range of motion of the at least one finger, number of repetitions between flexion and extension of the at least one finger and a pressure exerted by the at least one finger during flexion in memory, and determining one of compliance and progress of the rehabilitation based on the stored rehabilitation information.

In accordance with another embodiment of the present invention, a neurological device comprises a forearm support releasably attached to a user's arm, at least one finger sleeve adapted to be releasably attached to at least one finger, at least one tensor strut having a first end releasably coupled to the at least one finger sleeve and an opposite second end coupled to the forearm support, at least one sensor coupled to at least one of the at least one finger sleeve and the at least tensor strut, the at least one sensor configured to detect finger movement and generate electrical signals that are indicative of the movement, and a data device coupled to the sensor and configured to receive the electrical signals, and calculate at least one of a range of motion of the at least one finger, a speed of movement of the at least one finger, number of repetitions between flexion and extension of the at least one finger and a pressure exerted by the at least one finger during flexion from the electrical signals. Wherein the calculated data is used to remotely track user compliance and rehabilitation compliance by a healthcare provider.

Various combinations and sub-combinations of the disclosed elements, as well as methods of utilizing same, which are discussed in detail below, provide other objects, features and aspects of the present invention.

### BRIEF DESCRIPTION OF THE DRAWINGS

A full and enabling disclosure of the present invention, including the best mode thereof directed to one of ordinary skill in the art, is set forth in the specification, which refers to the appended figures, in which:

FIG. 1 is a perspective view of a neurological device in accordance with an embodiment of the present invention;

FIG. 2 is an exploded view of the neurological device of FIG. 1;

FIG. 3 is a perspective view of a neurological device in accordance with another embodiment of the present invention;

FIG. 4 is an exploded view of the neurological device of FIG. 3;

FIG. 5 is a top view of a neurological device in accordance with another embodiment of the present invention;

FIG. 6 is a side view of the neurological device of FIG. 5;

FIG. 7 is a perspective view of a neurological device in accordance with another embodiment of the present invention;

FIG. 7A is a partial perspective view of an alternate embodiment of a finger sleeve for use with the neurological device of FIG. 7;

FIG. 7B is partial perspective view an alternate embodiment of a finger glove for use with the neurological device of FIG. 7;

FIG. 7C is partial perspective view an alternate embodiment of a tensioner for use with the neurological device of FIG. 7;

FIG. 8 is a perspective view of a neurological device in accordance with another embodiment of the present invention;

FIG. 9 is a perspective view of a neurological device in accordance with another embodiment of the present invention; and

FIG. 10 is a perspective view of a neurological device in accordance with another embodiment of the present invention;

Repeat use of reference characters in the present specification and drawings is intended to represent same or analogous features or elements of the invention.

### DETAILED DESCRIPTION OF THE INVENTION

One of ordinary skill in the art will understand that the present discussion is a description of exemplary embodiments only, and is not intended as limiting the broader aspects of the present invention, which broader aspects are embodied in the exemplary construction. A repeat use of reference characters in the present specification and drawings represents the same or analogous features or elements of the invention.

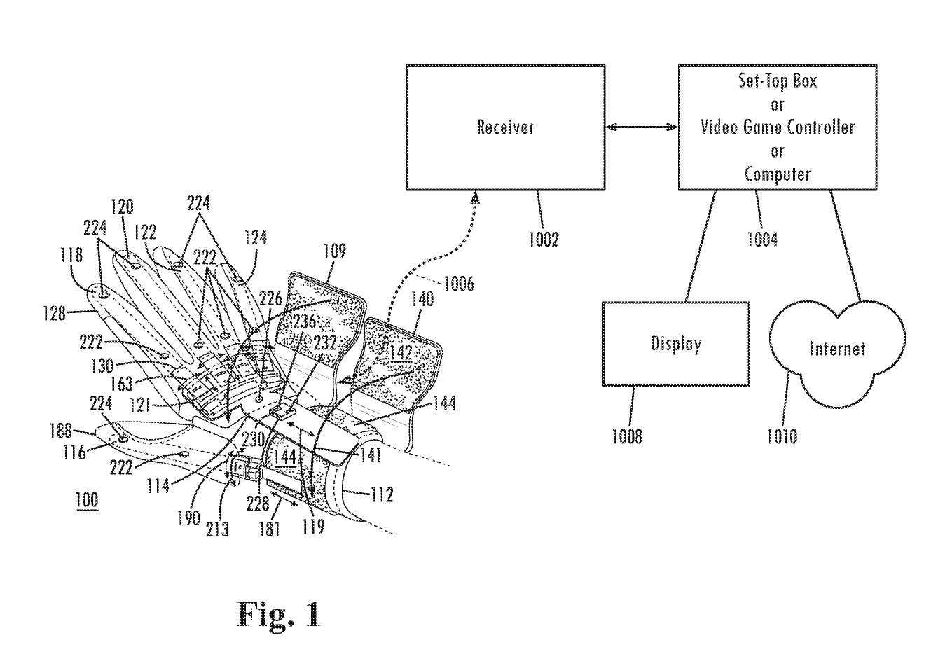

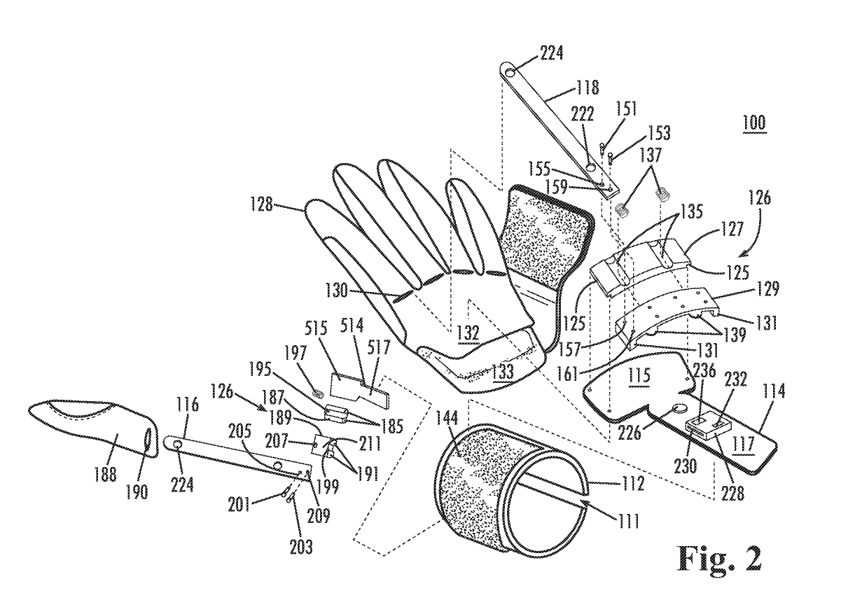

Referring to FIGS. 1 and 2, a neurological device 100 is shown having a forearm support section 112 and a hand support section 114 that are coupled together as described below. Forearm support section 112 is preferably formed from a flexible material such as plastic, metal, or alloy material. Forearm support section 112 also is configured and dimensioned to extend along a forearm of the user from the wrist rearwardly for a distance of at least several inches, and is generally tubular and designed to surround a portion of the wrist and forearm. Forearm support section 112 may be donned and doffed through an opening or slot 111 (FIG. 2) that extends the complete length of the forearm support section. Support section 112 is preferably lined with a permanent or removable close cell foam padded lining (not shown), and is adapted to tightly fit around the wrist and forearm with a frictional, interference fit. The lining may optionally include a non-skid material on the inner surface thereof to help prevent distal migration of forearm support section 112 along the user's arm. In one embodiment, forearm support section 112 is releasably secured on the user's forearm by an area of hooks 144 that is adapted to attach to an area of loops in conventional hook-and-loop attachment.

Hooks area 144 is preferably formed to substantially cover an outer surface of forearm support section 112 extending between the opposite ends that define slot 111. Hooks area 144 receives in hook-and-loop attachment areas of loops of a strap 140 (one such area 142 being shown in FIGS. 1 and 2). Strap 140 is preferably dimensioned and configured to extend substantially around forearm support section 112 to cover slot 111 and a base 117 (FIG. 2) of hand support section 114. Disposition of the covering attachment of strap 140 is illustrated by an arrow 141. To facilitate this, area 144 on forearm support section 112 is also adapted to receive, on a dorsal side thereof, a plurality of loops (not shown) disposed on an underside of hand support section 114 for removable attachment of base 117 to forearm support section 112. Additionally, area 144 is adapted to receive, on a radial side thereof, another plurality of loops (not shown), disposed on a thumb strut 116, for removeably attaching thumb strut 116 to forearm support section 112.

Hand support section 114 includes a platform 115, dimensioned and configured to extend between the radial side of the hand proximate the index finger across the back of the hand to little finger, and between the metacarpophalangeal joints and the carpals, i.e., between the base of the fingers and the wrist. Hand support section 114 further includes base 117 integrally formed with platform 115 and dimensioned and configured to extend across the length of forearm support section 112. Hand support section 114 preferably is constructed from a pliable, malleable material, e.g., a plastic or metal sheet that can be readily manipulated and shaped. That is, hand support section 114 preferably can be bent upward or downward at a juncture between platform 115 and base 117, as desired, to position the wrist at a selected one of a wide variety of angles when neurological device 100 is used to accommodate wrist flexion and/or extension. Thus, in use, hand support section 114 is preferably shaped so that the wrist is positioned upwardly as illustrated in FIG. 1.

A plurality of tension struts 118, 120, 122 and 124 are received in each respective finger of a glove 128 to provide tension between the fingers and hand support section 114. Each strut 118, 120, 122 and 124 is preferably constructed from, for example, spring steel and is formed with a thin or flat profile. Struts 118, 120, 122 and 124 are constructed to have varying degrees of resistance depending upon such factors as the thickness of the struts and materials from which the struts are made. Different resistances may be used with fingers having different characteristics of overall tone, tissue softness, and length. Each strut 118, 120, 122 and 124 corresponds in length and width to the finger to which it is attached. Suitable struts 118, 120, 122 and 124 may comprise, for example, thin resilient strips of about 0.01 to 0.008 inch stainless steel that is semi-rigid but nevertheless exhibits spring-like qualities.

Each strut 118, 120, 122 and 124 secured to a respective finger by inserting the strut in a respective elongate pocket 130 formed in each finger sleeve of glove 128. Each finger sleeve further is configured to enclose a respective one of the user's fingers, i.e., digit #2 through digit #5. Glove 128 includes a top surface 132 and a bottom portion 133. Each pocket 130 is preferably integrally formed in glove 128 during a conventional textile operation. Top surface 132 includes an area of loops (not shown) for attachment to an area of hooks (not shown) disposed on a bottom surface of platform 115. In should be understood that alternative attachment devices, such as snaps, buttons, zipper, buckles, etc. may be used to fasten the straps. In alternate constructions of glove 128, bottom surface 133 may be eliminated to provide an open palm construction.

Referring to FIG. 2, each strut 118, 120, 122 and 124 is releasably attached to hand support section 114, and specifically to platform 115, through an attachment mechanism 126 that is secured onto a top side of platform 115. Specifically, attachment mechanism 126 has a housing 127, which is secured to the platform top surface, and a slider 129, which mates with and slides, in directions designated by arrows 121 (FIG. 1), on top of housing 127. Slider 129 includes a C-shaped channel 131 on opposite sides that receive ledges 125 defined by housing 127, in interlocking engagement. Housing 127 further includes grooves 135 in which springs 137 are received and abut housing 127. Thus, when slider 129 is in interlocking engagement with the housing 127, one or more blocks 139, formed on an underside of slider 129, engage springs 137 and compress the springs when slider 129 moves away from base 117. Thus, springs 137 assist in opening the user's fingers by retracting the struts after the user makes a fist or closes their hand.

Each strut 118, 120, 122 and 124 mounts to slider 129 by two fasteners, such as screws 151 and 153. A first screw 151 extends through a curved slot 155 formed in the respective strut and is received in mating engagement within a threaded bore 157 in slider 129. A second screw 153 extends through a circular opening 159 formed in a respective strut and is received in mating engagement within another threaded bore 161 in slider 129. In this configuration, each respective strut is capable of rotational movement, in a respective direction designated by arrows 163, about second screw 153, with first screw 151 acting as a stop to define the limits of rotation. Moreover, either screw 151 and 153 may be tightened to lock the strut in a particular orientation.

A strut 116 for attachment to the user's thumb preferably is constructed from, for example, spring steel and is formed to have a thin or flat profile. Suitable struts may comprise, for example, thin resilient strips of about 0.01 to 0.008 inch stainless steel that is semi-rigid. Thumb strut 116 has a length and width that corresponds to the length and width of the user's thumb. Attachment of strut 116 to a thumb sleeve is achieved by insertion of the strut into an elongated pocket 190 formed in thumb sleeve 128. Thumb sleeve 128 is configured to enclose the user's thumb, and pocket 190 is preferably integrally formed in the glove. Strut 116 is releasably attached to forearm support section 112 through a thumb support section 514 (FIG. 2) that, similar to hand support section 114, includes a platform 515 and a base 517. An attachment mechanism 186 is secured on a top surface of platform 515 and functions to movably mount strut 116 to platform 515.

Base 517 of thumb support section 514 includes an area of loops (not shown) on a bottom surface thereof for releasably engaging with hook area 144 on forearm support section 112. Thumb support section 514, and in particular base 517, is configured and dimensioned to include a bend proximate the carpals of the wrist, which allows the thumb support to be bent to various degrees of flexion and extension at the carpals to allow the thumb to be positioned in varying degrees of thumb abduction, adduction, and opposition, depending on where attachment mechanism 186 is attached to thumb support section 514.

Referring again to FIG. 2, a slider 189 mates with and slides, in a direction designated by arrow 181 (FIG. 1), on top of housing 187. Slider 189 includes a C-shaped channel 191 on opposite sides thereof that receive side ledges 185, formed on housing 187, in interlocking engagement, in a similar manner to housing 127 and slider 129, as discussed above. Housing 187 further includes a groove 195 in which a spring 197 is received, which abuts housing 187 and, when slider 189 is in interlocking engagement with housing 187, a block 199 of slider 189 engages spring 197 and compresses it when slider 189 moves in a direction toward the thumb sleeve 188. Compression occurs when strut 116 is extended during closing of the hand, and spring 197 assists in opening of the hand by urging retraction of strut 116 and extension of the thumb.

Strut 116 is mounted to slider 189 by two fasteners, for example, screws 201 and 203. First screw 201 extends through a curved slot 205 formed in strut 116 and is received in mating engagement within a threaded bore 207. Second screw 203 extends through a circular opening 209 formed in strut 116 and is received in mating engagement within a threaded bore 211 in slider 189. In this configuration, strut 116 is capable of rotational movement, in the direction designated by arrow 213, about second screw 203, with first screw 201 acting as a stop defining the limits of such rotation.

A data device 228 is mounted on hand support section base 117 and comprises a processor (not shown), memory (not shown), a receiver (not shown), a transmitter (not shown), a secure digital (SD) slot 230, a USB port 232 and an antenna 236. Data device 228 communicates with a plurality of sensors 222, 224 and 226 located on neurological device 100. In particular, sensor 226 is positioned on hand support section 114 proximate data device 228 and may act as a reference for the other sensors. For each finger, sensors 222 are positioned proximate the proximal phalanxes, intermediate the user's knuckles and their proximal interphalangeal joints. Sensors 224 are positioned proximate to the user's distal phalanxes, intermediate the distal interphalangeal joints and the tips of the fingers. Sensors 222 may be coupled to glove 128 or attached to each respective strut 118, 120, 122, 124 and 116, as shown in FIGS. 1 and 2. With regard to the thumb, sensor 222 is positioned proximate the proximal phalanx, intermediate the knuckle and the distal interphalangeal joint. Sensor 224 is positioned proximate to the distal phalanx, intermediate the distal interphalangeal joint and the tip of the thumb. Similar to the finger sensors, the thumb sensors may be coupled to the thumb sleeve or directly attached to thumb strut 116, as shown in the figures.

It will be apparent to those skilled in the art that sensors 222, 224 and 226 may generate short range radio signals, which may be processed in accordance with public or proprietary processing circuitry and/or software. For example, communication of radio signals can be carried out using standards such as BLUETOOTH or other suitable wireless technology (e.g., such as IEEE 802.11). While it is preferred to employ technology not requiring line of sight, the embodiments described herein can be applied to technologies requiring line of sight such as infrared signals. Sensors 222, 224 and 226 may also be hardwired directly to data device 228. In either configuration, the sensors may contain one or more of a passive or active transceiver, accelerometers, strain gauges, pressure sensors, optical readers, potentiometers, etc. for detecting the movement of the sensors and the force applied to each sensor by the user.

The sensors are configured to detect the orientation of the fingers and thumb with respect to the user's palm, the speed the fingers move relative to one another and the user's hand and the pressure exerted by each finger on a real or virtual object. It is also contemplated that the sensors, or additional sensors distributed throughout the glove can provide tactile feedback to the user's fingers and thumbs to simulate the tactile feel of an object that the user is grasping in a virtual reality program.

In use, forearm support section 112 is first positioned and secured on the user's forearm, and hand support section 114 is shaped as desired to position the user's wrist relative to the forearm. In this respect, a healthcare worker, the user, or another person may bend hand support section 114 to achieve the desired angle for positioning of the wrist. Hand support section 114 is positioned or repositioned along the direction of arrows 119 on forearm support section 112 such that the bend in hand support section 114 is proximate to the user's wrist. A strap 109 may be fastened over the ends of struts 118, 120, 122 and 124 and attachment mechanism 126 for covering thereof. In this configuration, strap 109 includes an area of loops (not shown) for engagement with areas of hooks (not shown) formed on top surface 132. Thumb strut 116 is shaped and manipulated to position the thumb relative to forearm support section 112, and is attached to platform 515 of thumb support section 514. A strap 142 extends over and covers base 517 of thumb support section 514 including attachment mechanism 186 in its disposition on forearm support section 112.

Once attached, neurological device 100 creates rearwardly-directed forces that urge the fingers and thumb into an open hand position in which the fingers and thumb are extended. The resistance provided by each of the digit tensioners, i.e., each of tension struts 116, 118, 120, 122 and 124 is not so great as to prevent the user from moving their fingers and thumb towards a gripping position, thereby allowing the wearer to exercise (and rehabilitate) the hand. Neurological device 100 will generally position the user's wrist into extension with the digits extended, whereby the wearer will be in a position to grasp an object and, after grasping of the object, tension struts 116, 118, 120, 122 and 124 will assist in reopening the digits so the user will once again be in a position to grasp an object. Furthermore, each of the struts 116, 118, 120, 122 and 124 may be replaced by struts of different degrees of resilience, whereby the healthcare worker, the wearer, or another person can continue to select struts with the desired resistance for each digit as the healing and rejuvenation process progresses.

During rehabilitation, compliance and progress data is of great importance for ensuring compliance with the rehabilitation plan and shaping the rehabilitation process. To assist with compliance and rehabilitation planning, data device 228 is programmed to record the date, the start time and the end time for each occurrence that device is used. Data device 228 is also programmed to record all sensor data, and calculate progress and compliance data such as the number of times the user's hand is opened and closed, the range of motion and speed of each finger and thumb and the closing pressure exerted by the user's fingers when the fingers and thumb are moved into a grasping position. In this manner, a healthcare provider can use this information to determine both progress and compliance by the user.

Compliance information and progress information may be transmitted by data device 228 either wirelessly or via a wired connection 1006 to a receiver 1002 that is connected to a computing device 1004. Captured data can be manually or automatically transmitted via an internet connection 1010 from the computing device to the healthcare provider. In some embodiments, data device 228 may have its own designated IP address to allow the device to transmit the data over a wireless internet connection directly to the healthcare provider. In other embodiments, progress and compliance data may be transferred by way of an SD card received in SD slot 230 or by a USB connection through USB port 232. In all cases, the repetition data, range of motion data and closing pressure for each finger and thumb is transmitted to the healthcare provider to assist in providing a comprehensive up-to-date rehabilitation plan, as well as to support insurance billing through compliance data.

In addition to collecting rehabilitation progress and compliance data, data device 228 may also be configured to work interactively with computing device 1004 so as to function as a data input device. In this manner, a user of neurological device 100 can move their hand, wrist and fingers so that sensors 222 and 224 provide input signals that correspond to movement of the user's hand. Computing device 1004 is in communication with a display monitor 1010 so that the computing device transmits digital data to display 1010 to be viewed. Display 1010 may display text, menus and/or graphics, which show a virtual hand moving on the screen in relation to the user's movements, text indicating progress data or both. In particular, each of sensors 222 and 224 are configured to generate commands in response to a user's hand movements that are captured by data device 228 and transmitted to computing device 1004 through receiver 1002. The captured digital data enables neurological device 100 to be used as an interactive device with a computer program executed by computing device 1004. Thus, movement of a particular finger or fingers is transferred to computing device 1004 to initiate a command, response to a query, maneuver objects in an interactive video game, etc. Thus, the user can reach for and grasp virtual objects to assist in their rehabilitation without having to actually pick up or hold a physical object, which may be dangerous or difficult when the user lives alone or is home alone during a rehabilitation session. Use of neurological device 100 in conjunction with a virtual reality program or game also encourages the user to engage in rehabilitation exercises compared to just sitting and opening and closing their hand and fingers without interacting with a physical or virtual object.

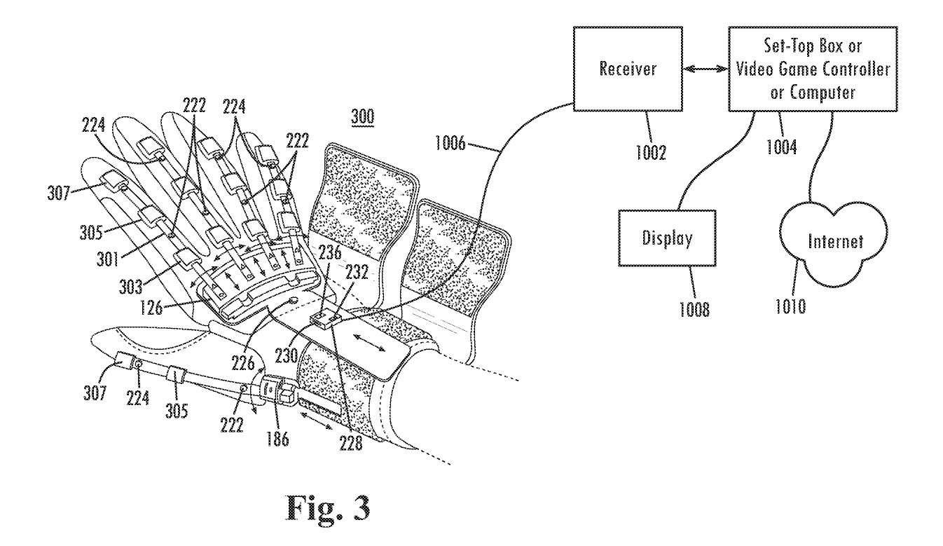

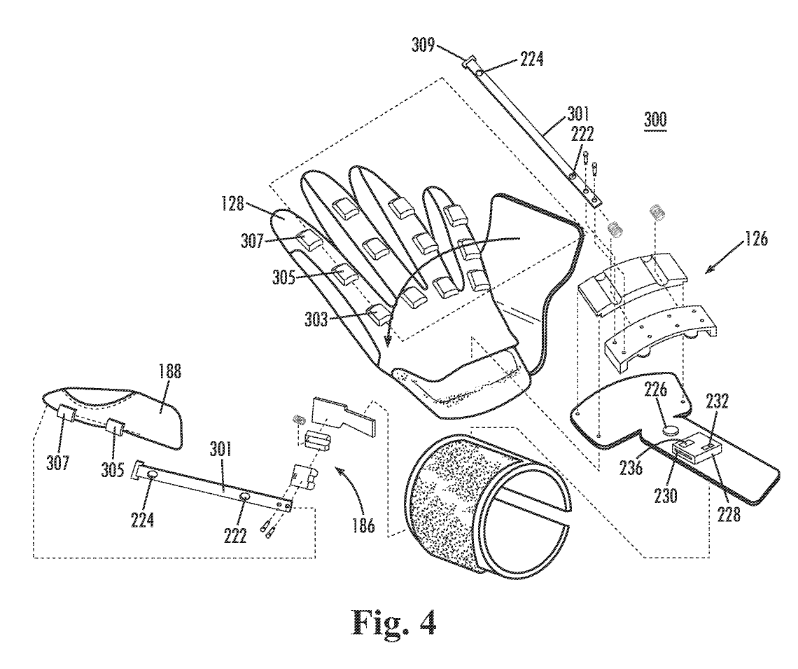

Referring to FIGS. 3 and 4, a second embodiment of a neurological device 300 is shown that is substantially similar to that shown in FIGS. 1 and 2. The main difference is how struts 301 are coupled to glove 128 and thumb sleeve 188. In particular, struts 301 are received in anchor guides 303, 305 and 307 attached along the fingers of glove 128 and thumb sleeve 188. Each strut 301 is formed with a cross-member 309 at a distal end thereof that abuts a respective top surface of anchor guide 307. In this manner, each end of strut 301 is axially fixed to anchor guide 307 in a rearward direction toward the user's wrist but is axially moveable away from the user's wrist. Similar to the embodiment shown in FIGS. 1 and 2, a plurality of sensors 222 and 224 are positioned along the user's fingers and thumb to allow movement of the digits to be sensed and tracked. Data device 228, as described above, receives sensor signals and transmits data via communications link 1006 to receiver 1002 and computing device 1004.

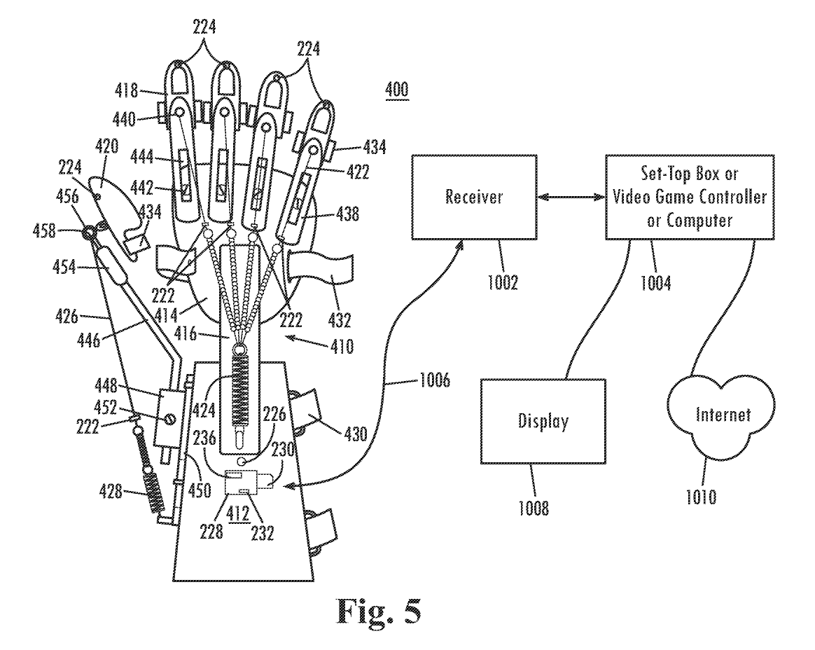

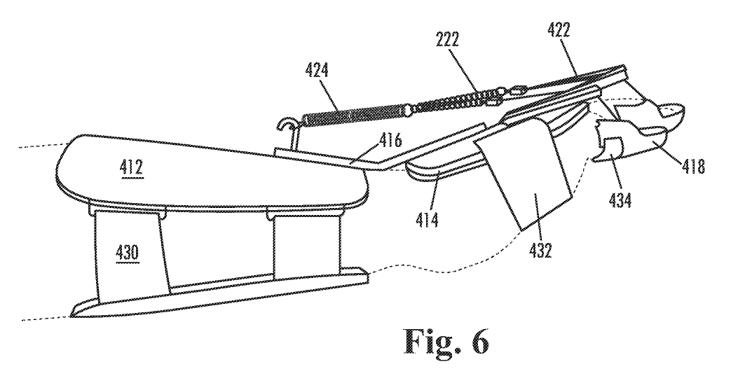

Referring to FIGS. 5 and 6, another embodiment of a neurological device 400 is shown having a forearm support 412, a hand support 414 and a support connector 416 that connects forearm support 412 and hand support 414 at an upward angle of approximately 25 to 45 degrees to raise the user's hand upwardly. A plurality of fingertip caps 418 are positioned over the tips of the user's fingers, while a thumb-tip cap 420 is positioned over the tip of the user's thumb. A plurality of releasable attachment straps 430, 432, 434 and 436, which include hook-and-loop type fasteners, is used to attach forearm support 412, hand support 414, finger tip caps 418 and thumb-tip cap 420 respectively to the user's forearm, hand and fingers.

Each fingertip cap 418 and thumb cap 420 contains a sensor 424 therein that detects movement of the user's fingers. Electronic components (not shown) may also be integrally formed in the finger and thumb caps that provide tactile stimulus to the user's fingers, as explained above. Sensors 424 may contain one or more of accelerometers, strain and pressure gauges, optical readers, potentiometers, etc that are configured to detect both movement and force applied by the user's fingers and thumb while moving the fingers and thumb into a grasping position. While sensors 424 are illustrated on the top of the finger caps, they may also be located on the underside of the finger caps.

A plurality of adjustable finger tension leads 422, having distal ends attached to fingertip caps 418, urge the fingertip caps from a gripping position to an open position. A proximal end of leads 422 are each attached to a finger tensioner 424, which in one preferred embodiment is a spring. Tensioner 424 is coupled at its proximal end to forearm support 412. Similarly, a thumb tension lead 426 has a distal end attached to thumb cap 420 and a proximal end attached to a lead 426, which is coupled to forearm support 412 by a tensioner 428. In a preferred embodiment, tensioner 428 is a spring that urges thumb-tip cap 20 from a gripping position to an open position. Each of tension leads 422 and tension lead 426 contain a sensor 422. Sensor 422 may be any type of sensor for measuring various characteristics, and in one preferred embodiment sensors 422 are strain gauges that detect the force applied to each tension lead 422 when the user moves their fingers and thumb into a gripping position.

Adjustable finger tension lead guides 438 are used to position fingertip caps 418 at the desired longitudinal and lateral locations in relation to hand support 414. Lead guides 438 have proximal ends adjustably attached to hand support 414 and distal ends including lead grommets or openings 440. Guides 438 may be adjusted longitudinally and rotatably to adjust the positions of openings 440. Adjustment is effected by an adjustment screw 442 that is positioned in a longitudinal slot 444. Each of finger tension leads 422 extends through a respective opening 440. In one preferred embodiment, sensors 422 may include an optical reader positioned adjacent a respective opening 440 and configured to read the movement of tension lead 438 passing through the opening.

A thumb tension lead guide 446, in the form of a bent rod, has a proximal end rotatable within a longitudinal bore (not shown) in a mounting block 448 that is supported on an adjustable base 450. A setscrew 452 in mounting block 448 is tightened against guide 446 once the guide is in the desired location. The longitudinal bore is aligned with a longitudinal axis of forearm support 412. A distal end of thumb tension lead guide 446 includes a threaded coupling nut 454 and thumbscrew 456 to longitudinally adjust guide 446. Thumbscrew 456 includes a bore 458, with thumb tension lead 426 extending through bore 458.

A data device 228, mounted on forearm support 412, is similar to that described above with respect to the embodiments shown in FIGS. 1-4, and communicates with sensors 222, 224 and 226 located on neurological device 400.

In operation, forearm support 412 is attached around the user's arm with hand support 414 being positioned on the back of the user's hand. Finger tip caps 418 are secured to the user's finger tips and thumb-tip cap 420 is secured to the user's thumb. Finger lead guides 438 are adjusted so that opening 440 is positioned approximately over finger tip caps 418. The distal end of lead 422 is attached to a respective one of finger tip caps 418 and strung through opening 440 in guide 438, and connected to spring tensioner 424. The lengths of leads 422 are adjusted to place leads 422 under tension, so that tensioner 424 urges leads 422 rearwardly and thereby urges the user's finger tips from a gripping position to an open position. It is important to note that the fingertip caps are axially fixed to the user's distal phalanxes above the distal interphalangeal joints to ensure that the user's hand is biased into the extended position.

Thumb tension lead guide 446 is rotatably positioned within mounting block 448 to a desired position and locked with setscrew 452, and thumbscrew 456 is positioned adjacent the desired location for thumb cap 420. The distal end of thumb tension lead 426 is attached to thumb-tip cap 420 and extends through bore 458 to thumb tensioner 428. The length of lead 426 is adjusted to place lead 426 under tension, so that tensioner 428 urges lead 426 rearwardly and thereby urges the user's thumb from a gripping position to an open position.

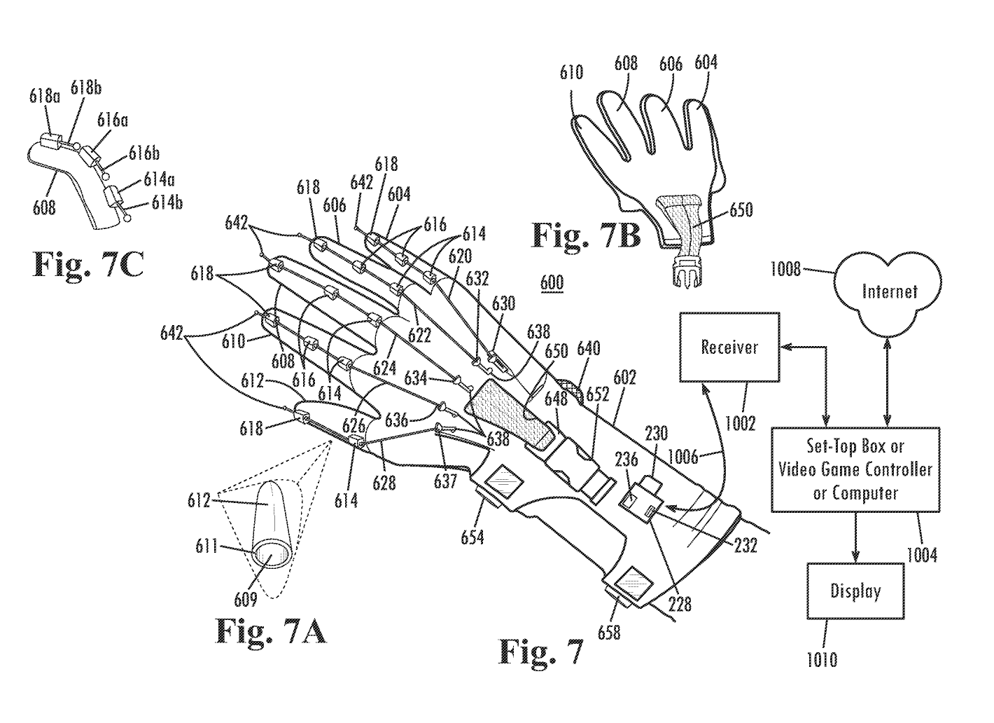

In yet another embodiment as shown in FIG. 7, a neurological device 600 has a forearm support section 602, a plurality of finger sleeves 604, 606, 608 and 610 and a thumb sleeve 612. Forearm support section 602 is dimensioned and configured to cover a portion of the user's forearm from the base of the hand to a point intermediate the wrist and the elbow. A portion of the forearm support section extends across the back of the hand between the wrist and the knuckles. Forearm support section 602 also includes one or more straps 654 and 658 for securing the forearm support section 602 in proper orientation. Straps 654 and 658 may include hook-and-loop fasteners such as VELCRO® fasteners. An inner surface of the forearm support section 12 is preferably lined with a padding material (not shown) for comfort.

Each finger sleeve 604, 606, 608 and 610 and a thumb sleeve 612 may be formed from a flexible, semi-rigid or rigid material, such as a textile, a polymer, an elastomer, etc. or some combination of these materials. Referring to FIG. 7A, the area surrounding an opening 609 in the sleeve may contain a rigid or semi-rigid ring 611. In other embodiments, rigid ring 611 may be formed as a semi-circle as opposed to a complete annular ring. The finger sleeves may be formed independently of one another, or in the alternative and referring to FIG. 7B, the finger sleeves may be formed integrally in a partial glove configuration. In this embodiment, a strap 650 includes one side of a buckle for securing the finger glove to forearm support section 612.

A plurality of tension struts 620, 622, 624, 626 and 628 are releasably coupled to a respective finger sleeve, on one side, and forearm support section 602, on the other side. Tension struts 620, 622, 624, 626 and 628 may be circular or oval in cross-section, semi-rigid, resilient rods formed from a hardenable mixture of filaments or fibers saturated in a resin, or can be made of any other resilient material with a suitable toughness to give a useful flexural fatigue life, such as advanced composite thermoplastics, thermosets, engineered plastics, fiber reinforced plastics, carbon fibers or ceramics. One preferred tension strut is formed from a matrix material of an epoxy or a resin and about 65 to 70 percent volume of S2-glass manufactured by Owens-Corning, thereby providing tension struts with an appropriate desired flexural strength. Each tension strut 620, 622, 624, 626 and 628 has a first bulbous end 638 that are received through respective openings 630, 632, 634, 636 and 637 formed in the portion of forearm support section 612 adjacent the back of the user's hand. A second bulbous end 642 is formed on an opposite end of the struts.

Each tension strut is slidably received within a respective plurality of tension strut slides positioned on a respective finger sleeve. In particular, each finger sleeve contains a first tension strut slide 614 coupled to a top surface of the sleeve and positioned proximate the finger proximal phalanx, intermediate the user's knuckle and the proximal interphalangeal joint. A second tension strut slide 616 is positioned proximate the finger intermediate phalanx, between the user's proximal interphalangeal joint and the distal interphalangeal joint. Finally, a third tension strut 618 is positioned proximate the finger distal phalanx, intermediate the user's distal interphalangeal joint and the tip of each finger. Thumb sleeve 612 includes two strut slides 618 and 614. The first, strut slide 614, is positioned adjacent the proximal phalanx, intermediate the knuckle and the thumb interphalangeal joint, and the second, strut slide 618, is positioned adjacent the distal phalanx, intermediate the tip of the thumb and the thumb interphalangeal joint.

Each tension strut slide 614, 616 and 618 may be passive in nature in that it merely provides a sliding guide for the strut, or it may be active in nature, in that it includes a linear encoder or other electrical sensor that generate signals indicative of the distance that the tension strut moves through the slide when the finger is moved from flexion to extension. The distance information can be collected and used, as described above to determine finger position and exerted finger and thumb pressure. In some embodiments, the tension struts may be removable in order to swap in a different strut that exerts a lower or higher amount of tension depending on the user's needs and rehabilitation plan.

In alternate embodiments as shown in FIG. 7C, tension struts 620, 622, 624, 626 and 628 may be replaced with hydraulic devices formed from a hydraulic cylinder 614a, 616a and 618a and respective hydraulic pistons 614b, 616b and 618b. The cylinders are mounted adjacent to the first, second and third phalanges on the finger sleeves. One end of the pistons are received in the respective hydraulic cylinder, and the other piston end is coupled to the finger sleeve. In this configuration, the fingers are pulled into the extension position. When the user moves their fingers into flexion, the pistons are partially pulled out of their cylinders against the hydraulic force. Once the user releases, the cylinders pull the fingers back into an extension position. In this configuration, sensors may be used to measure the pressure created as the pistons are pulled from the cylinders. In the alternative, linear encoders may be employed to measure the distance the pistons are pulled out of the cylinder. In either case, the generated signals may be used with a look-up table to determine the exerted force applied in the cylinder and distance that the piston moves.

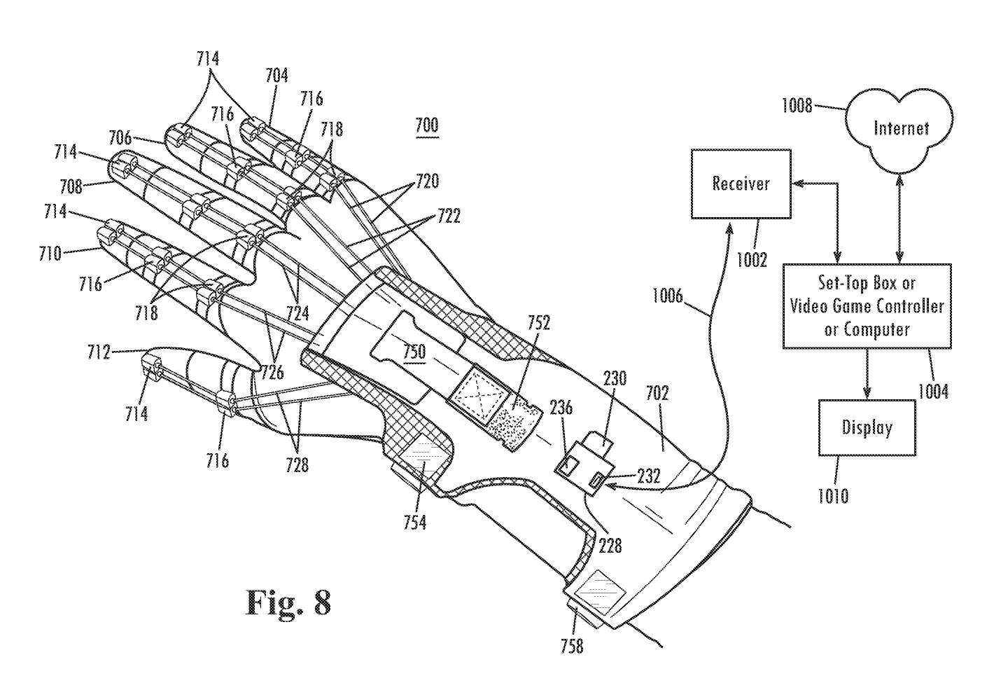

In yet another embodiment as shown in FIG. 8, n neurological device 700 has a forearm support section 702, a plurality of finger caps 704, 706, 708 and 710 and a thumb cap 712. Each finger cap 704, 706, 708 and 710 and thumb cap 712 may be formed from a flexible, semi-rigid or rigid material, such as a textile, a polymer an elastomer, etc. or some combination of these materials.

Forearm support section 702 is dimensioned and configured to cover a portion of the user's forearm from the base of the hand to a point intermediate the wrist and the elbow. A portion of the forearm support section extends across the back of the hand between the wrist and the knuckles. Forearm support section 702 also includes one or more straps 754 and 758 for securing the forearm support section 702 in proper disposition with respect to one another. Straps 754 and 758 may include hook-and-loop fasteners such as VELCRO® fasteners.

A plurality of tension struts 720, 722, 724, 726 and 728 are releasably coupled to a respective finger cap, on one side, and forearm support section 702, on the other side. Tension struts 720, 722, 724, 726 and 728 may be circular or oval in cross-section, semi-rigid, resilient rods formed from a hardenable mixture of filaments or fibers saturated in a resin, or can be made of any other resilient material with a suitable toughness to give a useful flexural fatigue life, such as advanced composite thermoplastics, thermosets, engineered plastics, or fiber reinforced plastics. In this particular embodiment, two struts are used for each finger.

One preferred tension strut is formed from a matrix material of an epoxy or a resin and about 65 to 70 percent volume of S2-glass manufactured by Owens-Corning, thereby providing tension struts with an appropriate desired flexural strength. Each tension strut is attached to a respective strut cap receptacle 714, at one end, and to the forearm support section at an opposite end. Each pair of tension struts for each finger are slidably received within a plurality of tension strut slides 716 and 718 that are releasably attached to the user's fingers by straps, elastic bands, etc. In particular, a first tension strut slide 716 is positioned proximate the finger proximal phalanx, intermediate the user's knuckle and the proximal interphalangeal joint, and a second tension strut slide 716 is positioned proximate the finger intermediate phalanx, intermediate the user's proximal interphalangeal joint and the distal interphalangeal joint. A single strut slide 716 is releasably secured to the thumb adjacent the proximal phalanx, intermediate the knuckle and the thumb interphalangeal joint.

Each strut slide 716 and 718 may be passive in nature in that it provides a slidable guide for the strut, or it may be active in nature, in that it includes a linear encoder or other electrical sensor that generate signals indicative of the distance that the tension strut moves through the slide when the finger is moved from flexion to extension. The distance information can be collected and used, as described above. In some embodiments, the tension struts may be removable in order to swap in a different strut that exerts a lower or higher amount of tension depending on the user's needs and rehabilitation plan.

A strap 750 is releasably secured to an area 752 of forearm support section 702. The releasable connection may be formed from any suitable structure such as a hook and loop fastener, snaps, buckles, etc. The releasable connection enables a user to adjust the angle of the user's wrist when wearing neurological device 700. A data device 228, mounted on forearm support section 702, operates substantially similar to that described above with respect to the other embodiments.

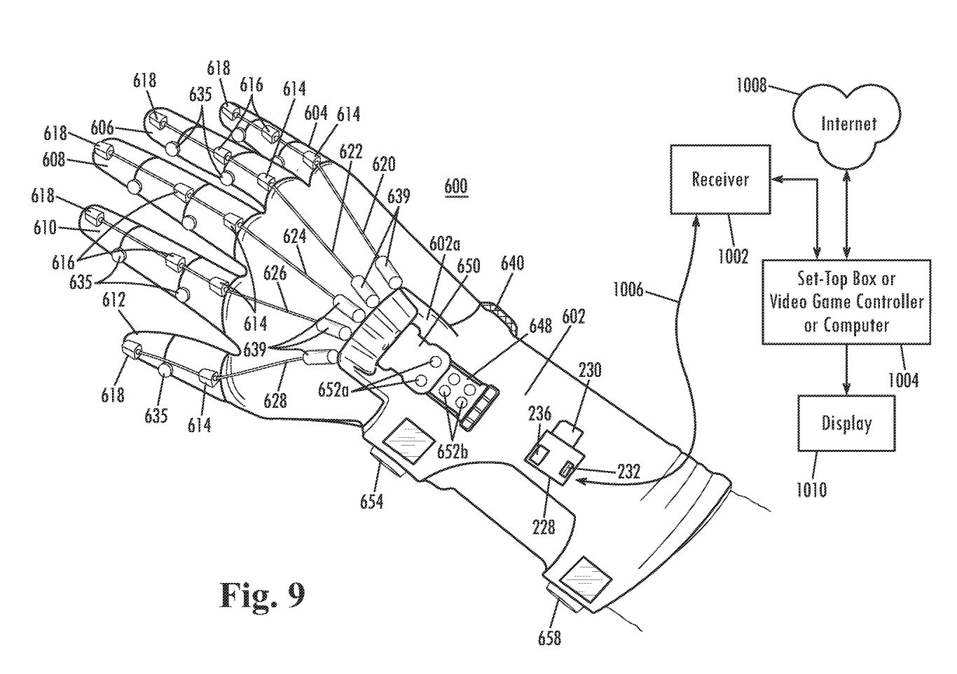

In yet another embodiment as shown in FIG. 9, a neurological device 600 is shown which is similar to the embodiment disclosed in FIG. 7. For purposes of brevity, only the differences between the two embodiments will be discussed herein. In the present embodiment, finger sleeves 604, 604, 608 and 610 are formed from a plurality of segments that are coupled together by a plurality of torsion springs 635 positioned at each joint. Torsion springs 635 provide a rotational bias that moves the fingers into a position of extension. Thus, the combination of torsion springs 635 and tension struts 620, 622, 624, 626 and 628 together function to bias the user's fingers and thumb into a position of extension. A plurality of strut slides 614, 616 and 618 are positioned along the length of each finger sleeve similar to that described above with respect to FIG. 7. Each strut slide may be passive, or it may contain a linear encoder to determine the position and movement of the finger. In addition, or instead of the linear encoders, potentiometers 639 are mounted on each tension strut. Potentiometer 639 may be in the form of a strain gauge or other suitable sensor for detecting the movement of the fingers and/or the force exerted by each finger during flexion.

A strap 650 is releasably secured to a strap 648 of forearm support section 602. The releasable connection may be formed from any suitable structure and in this embodiment the connection is carried out by snaps 652a and 652b. The releasable connection enables a user to adjust the angle of the user's wrist when wearing neurological device 600. A data device 228, mounted on forearm support section 602, operates substantially similar to that described above with respect to the other embodiments.

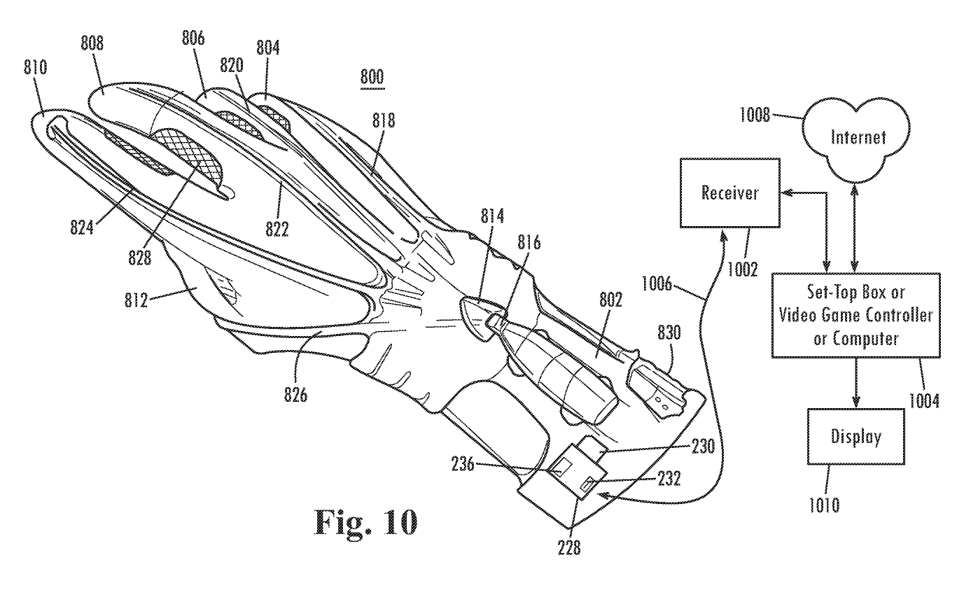

In yet another embodiment shown in FIG. 10, a neurological glove 800 is shown having a forearm support section 802 coupled to a plurality of finger sleeves 804, 806, 808 and 810 and a thumb sleeve 812. Support section 802, finger sleeves 804, 806, 808 and 810 and a thumb sleeve 812 may be integrally formed from a semi-rigid material such as a polymer or elastomer. A mesh textile portion 828 may be formed on the underside of each finger and thumb to releasably secure the hand, fingers and thumb to the neurological glove. A strap 830 may also be provided to secure forearm support section 802 to the user's forearm.

An air pressure port 814 having an air connection 816 is in fluid communication with a plurality of air channels 818, 820, 822, 824 and 826 that are respectively coupled to finger sleeves 804, 806, 808, 810 and thumb sleeve 812. Air connection 816 is configured to be releasably connected to a compressed air chamber, for example, a CO2 cartridge. Similar to the embodiments described above, a data device 228 may be included to receive and record data signals that are produced by sensors located along the fingers, thumb and on the hand portion of the neurological device. The sensors may be accelerometers, gyroscopes, pressure sensors etc. capable of detecting movement of the fingers and thumb. The data may be used to determine compliance and rehabilitation progress as discussed above.

In some embodiments, as shown in FIGS. 7-10, forearm support section 612 may be integrally formed with a hand support section (not separately numbered in FIGS. 7-10) in an area where the tension struts connect with the forearm support. In other embodiments, such as that shown in FIGS. 5 and 6, the hand support may be separately formed from the forearm support and attached thereto. In all embodiments, the hand support section is moveable with respect to the forearm support to assist in placing the wrist into a position of extension. In some embodiments, FIGS. 1-6, the hand support section is bendable with respect to the forearm support. In other embodiments, FIGS. 8-10, a strap may be used to move and retain the hand at an angle with respect to the forearm.

Referring to FIG. 7, this embodiment was described as having a strap 650 coupled to the forearm support by way of a two-piece buckle 648 and 652. In the alternative, or in combination, a hinged dial 640 may be used to change the angle of the hand support section with respect to the forearm support section. In particular, by rotating dial 640, the hand support may be moveable in discrete increments between zero and 60 degrees. Dial 640 may be placed on one or both sides of the hinge to allow the user or a third party to make angle adjustments. It is also contemplated that dial 640 may function as a locking mechanism where the hand support is rotatable with respect to the forearm support of a limited distance. By loosening dial 640, the user moves the hand support with respect to the forearm support, and when in the desired position, retightens dial 640 to maintain the desired angle. As indicated, a buckle, snaps, buttons or other fastening means may be used as a backup locking mechanism to ensure that the device does not accidentally loosen during use.

A method of capturing compliance and rehabilitation data comprises detecting the initial donning of the neurological device and recoding the data and time. Once the device is donned, data device 228 is activated and polls the sensors for finger movement data. As finger movement data is generated, the data is received by data device 228 and stored in memory in the data device. If the neurological device is being used in conjunction with a computer or video game controller, the signals are also passed to receiver 1002 via data connection 1006. Receiver 1002 transmits the data signals to computing device 1004, where the signals are converted into commands that move a virtual reality hand within a video program. The movement of the virtual reality hand may be displayed on display 1010 so the user can carry out various hand functions in virtual reality. The stored finger movement data in data device 228 may be converted into rehabilitation data by the data device. Once the rehabilitation session has ended, data device 228 may store the stop time so that the total rehabilitation session time may be computed. Rehabilitation data, such as range of motion, flexion pressure for each finger and thumb and repetition data, may be stored in the data device memory and/or transmitted via computing device 1004 over internet connection 1008 to the healthcare provider.

If the data is stored on the data device, it can be retrieved at a later time by an SD card or USB communication connection. The collected data may be used by the healthcare provider for monitoring rehabilitation and for future rehabilitation planning. The data may also be used by the healthcare professional for billing purposes since some insurance companies require the patient to comply with a rehabilitation plan in order for the insurance company to pay for the neurological device and provider services. The date may be used for these and other purposes related to rehabilitation or general exercise. Moreover, in some embodiments, the glove may be used merely as an input device to a computer program or game.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole and in part. Furthermore, those of ordinary skill in the art will appreciate that the foregoing description is by way of example only, and is not intended to limit the invention so further described in such appended claims. Therefore, the spirit and scope of the appended claims should not be limited to the description of the preferred versions contained therein.

What we claim is:

1. A neurological device comprising:
a. a forearm support that is configured to be releasably attached to a user's arm;
b. at least one finger sleeve adapted to be releasably attached to at least one finger;
c. at least one tensor strut having a first end releasably coupled to said at least one finger sleeve and an opposite second end coupled to said forearm support;
d. at least one sensor coupled to at least one of said at least one finger sleeve and said at least tensor strut, said at least one sensor configured to detect finger movement and generate electrical signals that are indicative of said movement;
e. a data device coupled to said sensor and configured to:
i. receive said electrical signals;
ii. calculate at least one of a range of motion of said at least one finger, a speed of movement of said at least one finger, number of repetitions between flexion and extension of said at least one finger and a pressure exerted by said at least one finger during flexion; and
iii. store said at least one of a range of motion of said at least one finger, a speed of movement of said at least one finger, number of repetitions between flexion and extension of said at least one finger and a pressure exerted by said at least one finger during flexion in memory,
wherein said stored data is used to track user compliance and rehabilitation compliance by a healthcare provider.

2. The neurological device of claim 1, further comprising:
a. a plurality of finger sleeves adapted to be releasably attached to a respective finger of the user;
b. a thumb sleeve adapted to be releasably attached to the thumb of a user;
c. a plurality of tension struts, each releasably coupled to a respective one of said plurality of finger sleeves and said thumb sleeve; and
d. a plurality of sensors, each of said plurality of sensors being operatively coupled to at least one of said plurality of finger sleeves and said thumb sleeve and said plurality of tensor struts,
wherein each of said plurality of sensors are operatively coupled to said data device.

3. The neurological device of claim 2, wherein said plurality of finger sleeves are integrally formed with one another to form a partial glove.

4. The neurological device of claim 1, wherein said at least one sensor is wirelessly connected to said data device.

5. The neurological device of claim 1, wherein said at least one sensor is wired to said data device.

6. The neurological device of claim 1, wherein said data device further comprises at least one of a USB port, an SD card slot and an antenna.

7. The neurological device of claim 1, wherein
said finger sleeve is formed from a plurality of segments, and
at least one torsion spring coupled to adjacent segments.

8. The neurological device of claim 1, further comprising a plurality of couplers that releasably attach said at least one tension strut with said at least one finger sleeve.

9. The neurological device of claim 1, wherein said tension strut is formed from one of a carbon fiber rod, a fiber reinforced polymer, a hydraulic piston and an elastomer band.

10. The neurological device of claim 1, wherein said data device is operatively coupled to a computing device through a data receiver so that said neurological device is used as an input device to said computing device for making data entries and responding to queries.

11. The neurological device of claim 10, wherein said computing device is running a virtual reality program that allows the user to interact with said program by making finger and hand movements with said neurological device.

12. The neurological device of claim 1, wherein said tensor strut second end is coupled to said forearm support by a fastener.

13. The neurological device of claim 12, wherein said fastener is one of a adjustable buckle, a set of snaps, buttons, zipper and hooks and loops.

14. The neurological device of claim 1, wherein said finger sleeve is configured to extend from a tip of the finger to a point intermediate the finger tip and a distal interphalangeal joint.

15. The neurological device of claim 14, further comprising a plurality of tension strut slides positioned intermediate said tension strut first and second ends intermediate said finger sleeve and said forearm support, wherein each of said plurality of tension strut slides is releasably attached to the user's finger.

16. The neurological device of claim 1, further comprising an air pneumatic connector having a pneumatic port, said tension strut comprising an air passage that is in fluid communication with said finger sleeve and said pneumatic port, wherein said pneumatic port is configured to receive compressed air.

17. The neurological device of claim 1, further comprising a hand support section intermediate said forearm support section and said tensor strut second end.

18. The neurological device of claim 17, wherein said hand support section is movable with respect to said forearm support section over a range of angles.

19. A method of collecting rehabilitation compliance and progress data, comprising the steps of:
a. providing a neurological device having:
i. a forearm support that is configured to be releasably attached to a user's arm;
ii. at least one finger sleeve adapted to be releasably attached to at least one finger;
iii. at least one tensor strut having a first end releasably coupled to said at least one finger sleeve and an opposite second end coupled to said forearm support;
iv. at least one sensor coupled to at least one of said at least one finger sleeve and said at least one tensor strut, said at least one sensor configured to detect finger movement and generate electrical signals that are indicative of said movement; and
v. a data device coupled to said sensor,
b. receiving said electrical signals;
c. calculating at least one of a range of motion of said at least one finger, a speed of movement of said at least one finger, number of repetitions between flexion and extension of said at least one finger, a pressure exerted by said at least one finger during flexion and date and time;
d. storing said at least one of a range of motion of said at least one finger, number of repetitions between flexion and extension of said at least one finger and a pressure exerted by said at least one finger during flexion in memory; and
e. determining one of compliance and progress of said rehabilitation based on said stored rehabilitation information.

20. A neurological device comprising:
a. a forearm support releasably attached to a user's arm;
b. at least one finger sleeve adapted to be releasably attached to at least one finger;
c. at least one tensor strut having a first end releasably coupled to said at least one finger sleeve and an opposite second end coupled to said forearm support;
d. at least one sensor coupled to at least one of said at least one finger sleeve and said at least tensor strut, said at least one sensor configured to detect finger movement and generate electrical signals that are indicative of said movement;
e. a data device coupled to said sensor and configured to:
i. receive said electrical signals; and
ii. calculate at least one of a range of motion of said at least one finger, a speed of movement of said at least one finger, number of repetitions between flexion and extension of said at least one finger and a pressure exerted by said at least one finger during flexion from said electrical signals; and
wherein said calculated data is used to remotely track user compliance and rehabilitation compliance by a healthcare provider.
